(19) United States
(12) Patent Application Publication
(10) Pub. No.: US 2016/0184219 A1
(43) Pub. Date: Jun. 30, 2016

(54) Use of Genetically Modified Skin Flora for Body Odor Relief in Intertriginous Areas

(71) Applicant: Kevin Matthew Glass, Mountain View, CA (US)

(72) Inventor: Kevin Matthew Glass, Mountain View, CA (US)

(21) Appl. No.: 14/583,887

(22) Filed: Dec. 29, 2014

(51) Int. Cl. A61K 8/99; A61Q 15/00

(52) U.S. Cl. CPC A61K 8/99; A61Q 15/00

(57) ABSTRACT

The use of genetically modified skin flora in intertriginous regions of skin to manage body odor. To manage body odor, said skin flora would secrete various essential oils in order to alter the scent. These essential oils would be produced via metabolic pathway, the enzymes of which would be encoded in plasmids added to the intertriginous skin flora via transformation.

### CROSS-REFERENCE TO RELATED APPLICATIONS

US Utility Patent Provisional Application 61/921,511, filed 29 Dec. 2013 by Kevin Glass.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### REFERENCES CITED

U.S. Patent Documents

| | | |
|---|---|---|
| 8,772,354 | Williams, et al. | July 2014 |
| 8,895,040 | Vondruska, et al. | November 2014 |

Foreign Patent Documents

| | | |
|---|---|---|
| DE 200810059356 | Ruediger, Braun | November 2008 |

### Other References (Non-Patent Literature)

Dixon, James, and Natalie Kuldell. “Using Banana-Scented Bacteria to Teach Synthetic Biology.” Methods for Part/Device Characterization and Chassis Engineering. Ed. Christopher Voigt. Vol. 497. San Diego, Calif.: Academic, 2011. 255-71. Print.

### BACKGROUND OF THE INVENTION

The present invention is in the technical field of body odor management.

More particularly, the present invention is in the technical field of intertriginous body odor management. More particularly, the present invention is applicable to axillary and groinal intertriginous regions. Intertriginous referring to areas of the body where two skin areas make contact.

Traditional methods of body odor management are usually either reliant on antiperspirants, antimicrobial agents, or substances that emit and diffuse a pleasing, agreeable scent. U.S. Pat. No. 8,772,354 describes the use of malodor counteracting compositions to suppress body odor, using various organic compounds to suppress body odor. The invention herein is the use of genetically modified bacteria with the object of body odor management or suppression. Often these compounds require repeated use and can be costly and ineffective. These chemical remedies can sometimes fail persons with acute body odor, and thus be only partially effective. Said previous body odor management methods attempt to remedy odor by means of suppressing or killing naturally occurring skin flora that produce said odor.

Other art using genetically modified skin flora have utilized skin flora modified in different ways. Published foreign (German) patent application DE 102008059356 describes the use of gram positive bacteria to enhance skin flora within the object of preventing acne. Said art uses genetically modified or naturally occurring bacteria to prevent acne by means of forming a protective layer on the skin. The body odor management system claimed herein utilizes genetically modified bacteria to alleviate body odor within intertriginous areas of the skin. The bacteria within the claimed invention are modified to synthesize esters, essential oils, antimicrobial agents, or other compounds for the purpose of body odor suppression. Both the object and modification of the bacteria claimed herein are different that the aforementioned art.

Art describing the use of esters for personal care products: U.S. Pat. No. 8,895,040 utilizes a direct external application of esters as personal care products. In contrast the invention claimed herein describes genetic modification of skin flora to secrete esters to suppress body odor, and does not include the application of esters directly on the skin. Instead the body odor management system embodied herein uses genetically modification of skin flora to secrete compounds with the object of body odor suppression, not limited to the esters, whose use is claimed by prior art.

Prior art of genetically modifying bacteria to produce a specific scent (Dixon and Kuldell, 2011), has used a bacterial conversion of isoamyl alcohol to isoamyl acetate via genetic modification. The prior art utilizes external chemicals to accomplish the object of their system, which has been within a controlled laboratory environment. The invention embodied herein utilizes genetically modified bacteria to suppress odors that are not specific to the modified bacteria, and, in addition to producing a pleasant fragrance using chemicals already extant on the skin, produces anti-microbial substances to regulate the growth of other odor causing bacteria. Furthermore, the embodied invention is not limited to a specific technique of modification but is more broadly a method of managing interegionous body odor vis-à-vis genetically modified skin flora, which, in addition to bacteria, can be fungi. Furthermore, the embodied invention utilizes varied secretion of substances via a feedback loop, using several different genetically modified varieties of skin flora for different functions.

### SUMMARY OF THE INVENTION

The present invention is the use of genetically modified skin flora to manage body odor. This method of body odor management uses the skin flora that produce said odor in intertriginous regions to instead produce a pleasant fragrance via production of essential oils. These would be automatically produced after the initial introduction of the modified bacteria.

### BRIEF DESCRIPTION OF THE DRAWING

FIG. 1 is a visual representation of the invention.

### DETAILED DESCRIPTION OF THE INVENTION

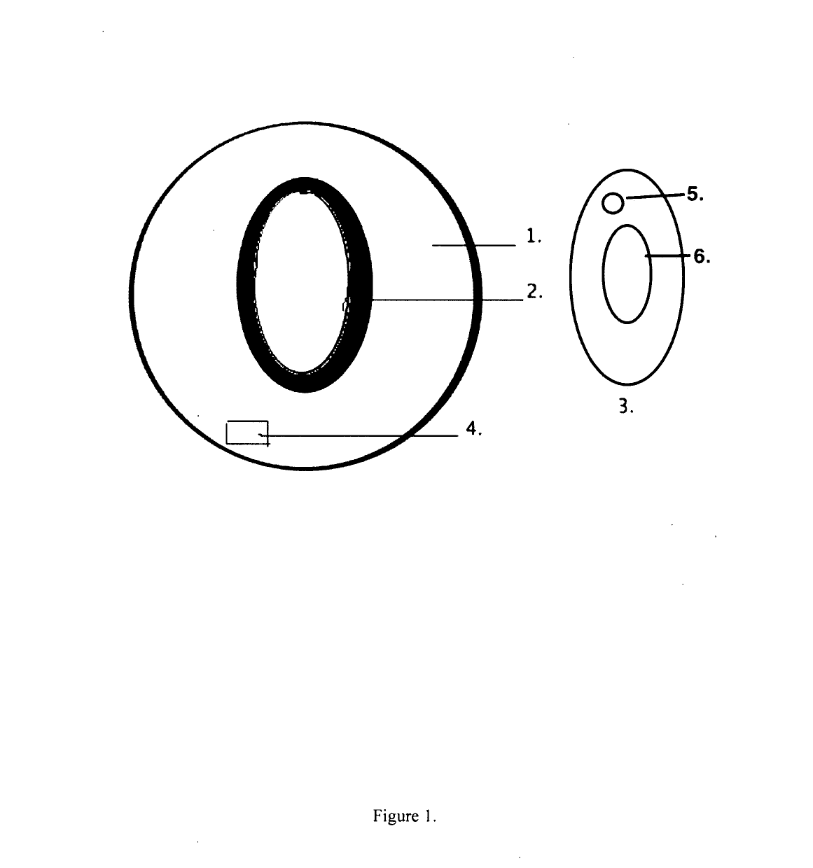

Referring now to the invention in more detail, in FIG. 1 there is shown a genetically modified skin flora 1 in an intertriginous area of skin 3 using an inserted plasmid 2 to produce enzymes 4 that produce the essential oil. This can be done via several methods for inserting plasmids into bacteria, such as through heat shock transformation.

In more detail, still referring to the invention of FIG. 1, the genetically modified skin flora 1 would secrete various compounds and oils in order to alter body odor. In further detail, still referring to the invention of FIG. 1 the skin flora must be modified with a specific gene encoding the metabolic pathway to produce said essential oil (for example Geraniol or Nerol, two essential oils that are produced via biosynthetic pathway) via several plasmid vectors 2. Said modified skin flora would also have to be placed in an intertriginous environment 3 in order to survive and compete successfully with other non-modified skin flora.

The production details of the invention, as shown in FIG. 1, are that the modified bacteria may be any bacteria that can be successfully modified via plasmid vector 3 that can survive in the environment and is nonpathogenic (good examples include benign modified staphylococci bacteria).

The advantages of the present invention include, without limitation, the convenience of use of not having to apply daily, which one would have to do with other body odor management techniques, as well as the lack of the skin irritation or dermatological damage caused by traditional methods of body odor management. Other advantages include the increased ease of use of the product, as it would only require a single use and from then on would not require repeated application; the ability to suppress the introduced scent via traditional odor management methods; and the positive effect of having the secreted compounds be non staining and easily washed out.

In broad embodiment, the present invention is the use of genetically modified skin flora using essential oils to manage body odor by creating a pleasant fragrance. While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A method of body odor management, comprising:
(a) providing skin-compatible flora which have been modified to secrete substances effective in improving body odor, and
(b) applying said flora to skin surfaces,
whereby body malodor will be suppressed with reduced skin irritation and lasting longer between applications than the prior art.

2. The method of body odor management of claim 1 further including that said flora are genetically modified.

3. The method of body odor management of claim 2 further including that said flora are modified using plasmid insertion.

4. The method of body odor management of claim 3 further including that said flora are modified with a specific gene or set of genes encoding their metabolic pathway.

5. The method of body odor management of claim 4 further including that said metabolic encoding will cause said flora to secrete various compounds and oils effective in altering body odor.

6. The method of body odor management of claim 3 wherein said flora are modified by means of plasmid vector.

7. The method of body odor management of claim 6 wherein said plasmid vector contains a promoter, a gene sequence, and a selective marker.

8. The method of body odor management of claim 1 wherein applying said flora is performed to intertriginous regions.

9. The method of body odor management of claim 1 wherein said flora utilize human secretions in their metabolism.

10. The method of body odor management of claim 1 further including that said flora produce secondary metabolites that produce substances that suppress odor.

11. The method of body odor management of claim 1 wherein said flora utilize externally controlled feedback loops to produce substances that directly or indirectly control body odor.

12. The method of body odor management of claim 11 wherein the said body odor management system contains a substance that interacts with biofeedback loops of the said flora, as a means to control the expression of enzymes by the said flora.

13. The method of body odor management of claim 12 further including that said enzymes directly synthesize or facilitate the production by the said flora of substances that suppress body odor.

14. The method of body odor management of claim 11 wherein said flora are modified to produce anti-microbial agents in response to external stimuli.

15. The method of body odor management of claim 1 wherein said flora are genetically modified to be resistant to the odor suppressing substances produced by said flora.

16. The method of body odor management of claim 1 wherein said body odor management system is removable by means of a safe solvent.

17. The method of body odor management of claim 1 wherein said flora undergo lysis when exposed to detergents.

18. The method of body odor management of claim 1 wherein said flora are attenuable by means of electric charge.

19. The method of body odor management of claim 1 wherein said flora are susceptible to specific antimicrobial agents.

20. The method of body odor management of claim 1 wherein said flora are genetically modified for production of different chemicals depending upon environmental conditions.

21. A body deodorant, comprising:
(a) skin-compatible flora which have been modified to secrete substances effective in improving body odor, and
(b) means for application of said flora to skin surfaces,
whereby pleasing body odors will be created with reduced skin irritation and lasting longer between applications than the prior art.

22. The deodorant of claim 21 further including that said flora are genetically modified.

23. The deodorant of claim 22 further including that said flora are modified with a specific gene encoding their metabolic pathway.

24. The deodorant of claim 23 further including that said metabolic encoding will cause said flora to secrete various compounds and oils effective in altering body odor.

25. The deodorant of claim 21 wherein applying said flora is performed to intertriginous regions.

26. The deodorant of claim 21 wherein said flora utilize externally controlled feedback loops to produce substances that directly or indirectly control body odor.
